(19) United States
(12) Patent Application Publication
(10) Pub. No.: US 2007/0254003 A1
(43) Pub. Date: Nov. 1, 2007

(54) Non-sticky coatings with therapeutic agents for medical devices

(76) Inventor: Pu Zhou, Maple Grove, MN (US)

Correspondence Address: JONES DAY, 222 EAST 41ST ST, NEW YORK, NY, 10017, US

(21) Appl. No.: 11/416,488

(22) Filed: May 1, 2006

(51) Int. Cl. A61K 31/4745; A61K 31/337; A61F 2/02

(52) U.S. Cl. 424/423; 514/291; 514/449

(57) ABSTRACT

A stent for delivering a therapeutic agent to body tissue of a patient and a method of making such a medical device are disclosed. The invention relates generally to a medical device such as an intravascular stent that is useful for delivering a therapeutic agent to the body tissue of a patient, and the method for making such a device. More particularly, in one embodiment the invention is directed to a stent having a tubular sidewall comprising a plurality of struts, in which the inner and outer surfaces of the struts are coated with different coating compositions. In another embodiment, a first coating composition comprising a therapeutic agent and a first polymer is disposed on the outer surface of the strut, while the inner surface is free of any coating composition.

### FIELD OF THE INVENTION

The invention relates generally to a medical device such as an intravascular stent that is useful for delivering a therapeutic agent to the body tissue of a patient, and a method for making such a device. More particularly, in one embodiment the invention is directed to a stent having a sidewall structure comprising a plurality of struts, in which the inner and outer surfaces of the struts are coated with different coating compositions. In another embodiment, a first coating composition comprising a therapeutic agent and a first polymer is disposed on the outer surface of the strut, while the inner surface is free of any coating composition.

### BACKGROUND OF THE INVENTION

Medical devices, such as implanted stents, have been coated with compositions comprising a therapeutic agent. One method of applying coatings loaded with a therapeutic agent to stents and other medical devices having a tubular portion is to coat the inside (adluminal surface), sides, and outside (abluminal surface) of the tubular portion of the medical device with the composition to form a continuous coating on the tubular portion. A reason for coating all these surfaces of the tubular portion of the medical device with a coating is to ensure adherence of the applied coating to the tubular portion. For example, when the tubular portion is comprised of struts, applying a coating composition to all surfaces of the struts will form a coating that wraps around the struts. The fact that the coating “wraps around” the struts enhances adherence of the coating to the tubular portion.

However, in many stents all of the surfaces of the medical device or portions thereof do not need to be coated with a coating composition comprising a therapeutic agent. For instance, in a vascular stent, the inner surface and side surfaces of the tubular portion may not have to be coated with a coating composition containing a therapeutic agent. This is because these parts of the stent do not come in direct contact with the body lumen wall and do not apply the therapeutic agent to the body lumen wall. Therefore, it is not necessary to coat the inner surface and sides of the stent struts with a coating composition containing a therapeutic agent that is being applied to the body lumen wall.

Moreover, in order to deliver certain stents, such as a balloon expandable stent, comprising a sidewall having struts, the stent must be put in its unexpanded state or “crimped” before it is delivered to a body lumen. Crimping can cause the coating composition to be torn or ripped off the struts. Specifically, if the first coating composition that is applied to the side surfaces of the struts of the stent contains a polymer that is relatively soft or tacky, then the coating composition will have a tendency to adhere to the side surfaces of adjacent struts during the crimping process. Such adherence will cause the coating composition to be ripped off the surfaces when the stent is expanded. Also, if the coating composition that is applied to the inner surface of the struts, which contacts the balloon, is coated with a material that is relatively soft or tacky, such coating will tend to be ripped off the inner surface because the coating will stick to the balloon as it contacts the inner surface during expansion. Therefore, there are problems associated with using relatively soft polymers in coatings. However, to form a coating containing a therapeutic agent, it is desirable to use such relatively soft polymer because such materials have a better ability to incorporate the therapeutic agent.

Accordingly, there is a need for more efficient methods of coating a stent having a sidewall comprised of struts, that can more accurately deliver the desired dosage of a therapeutic agent from the coating of the device in order to limit patient exposure to excess drug in the coating. Furthermore, there is a need for a coated expandable stent comprising struts in which the undesired removal of coating from the stent is minimized.

### SUMMARY OF THE INVENTION

These and other objectives are accomplished by the present invention. In one embodiment, the invention is directed to an intravascular stent, such as a balloon-expandable stent, comprising a metal stent sidewall structure designed for implantation into a blood vessel of a patient. The sidewall structure comprises a plurality of openings therein and struts each having an outer surface (abluminal surface) and an inner surface (adluminal surface) opposite the outer surface. There is a first coating composition disposed on at least a portion of the outer surface of at least some of the struts. The first coating comprises a therapeutic agent, such as an anti-restenosis agent, and a first polymer, such as a biostable polymer. The inner surface of each of the plurality of struts is free of any coating composition and the first coating composition conforms to the outer surface of at least one strut to preserve the openings of the stent sidewall structure.

In some embodiments, the struts comprise at least one side surface adjacent to the outer surface and the inner surface, wherein the inner surface and outer surface are connected by the side surface. In one embodiment, the side surface of each of the plurality of struts is free of any coating composition. In other embodiments, the first coating composition is disposed on at least a portion of the side surface of at least some of the struts. In another embodiment, the first coating composition conforms to the outer surface and the side surface to preserve the openings of the stent sidewall structure.

In some embodiments, the therapeutic agent comprises an anti-thrombogenic agent, anti-angiogenesis agent, anti-proliferative agent, anti-restenosis agent, growth factor, radiochemical or antibiotic. In alternative embodiments, the therapeutic agent comprises paclitaxel, sirolimus, everolimus, tacrolimus, or pimecrolimus.

In other embodiments, the first polymer is biostable. Generally, the first polymer can comprise a styrene-isobutylene copolymer, polyurethane, silicone, polyester, polyolefin, polyisobutylene, ethylene-alphaolefin copolymer, acrylic polymer or copolymer, vinyl halide polymer, polyvinyl ether, polyvinylidene halide, polyacrylonitrile, polyvinyl ketone, polyvinyl aromatic, polyvinyl ester, copolymer of vinyl monomers, copolymer of vinyl monomers and olefins, polyamide, alkyd resin, polycarbonate, polyoxymethylene, polyimide, polyether, epoxy resin, polyurethane, rayon-triacetate, cellulose, cellulose acetate, cellulose butyrate, cellulose acetate butyrate, cellophane, cellulose nitrate, cellulose propionate, cellulose ethers, carboxymethyl cellulose, collagen, chitin, polylactic acid, polyglycolic acid, polylactic acid-polyethylene oxide copolymer, EPDM rubber, fluorosilicone, polyethylene glycol, polysaccharide, or phospholipid. In some embodiments, the stent sidewall structure is balloon expandable. In other embodiments, the stent sidewall structure comprises a metal.

In alternative embodiments, the invention is directed to an intravascular stent, such as a balloon-expandable stent comprising a metal stent sidewall structure designed for implantation into a blood vessel of a patient, wherein the stent sidewall structure comprises a plurality of openings therein and struts each having an outer surface (abluminal surface) and an inner surface (adluminal surface) opposite the outer surface. There is a first coating composition disposed on at least a portion of the outer surface and the inner surface of at least some of the struts, wherein the first coating comprises a therapeutic agent, such as an anti-restenosis agent, and a first polymer, such as biostable polymer. There is a second coating composition disposed on the first coating composition that is disposed on the inner surface of at least one strut. The second coating composition is not disposed on the first coating composition that is disposed on the outer surface of the struts. The second coating composition comprises a second polymer, such as biostable polymer that has less tackiness than the first polymer and is free of any therapeutic agent when applied to the outer and inner surfaces.

In certain embodiments, the struts comprise at least one side surface adjacent to the outer surface and the inner surface and connects the inner surface and outer surface. In some embodiments, the side surface is free of any coating composition. In other embodiments, the second coating composition is disposed on at least a portion of the side surface of at least some of the struts and the first coating composition is not disposed on the side surface of any of the struts. In alternative embodiments, the first coating composition is disposed on at least a portion of the side surface of at least some of the struts and the second coating composition is not disposed on the first coating composition disposed on the side surface of any of the struts. In still other embodiments, the first coating composition is disposed on at least a portion of the side surface of at least some of the struts and the second coating composition is disposed on the first coating composition that is disposed on the side surface of the struts. In some embodiments, the first coating composition conforms to the outer surface and inner surface of the struts to preserve the openings of the stent sidewall structure and the second coating composition conforms to the inner surface of the at least one strut to preserve the openings of the sidewall structure.

In certain embodiments, the second polymer is harder than the first polymer. In other embodiments, the second polymer has a hardness of greater or more than about 40 A. In particular embodiments, the second polymer has a tackiness of less than about 50 g, such as about 3 g to about 30 g. In some embodiments, the first polymer has a tackiness of more than about 50 g. In certain embodiments, either or both of the first polymer and second polymer are biostable. In one embodiment, the polymer comprises a styrene-isobutylene copolymer, polyurethane, silicone, polyester, polyolefin, polyisobutylene, ethylene-alphaolefin copolymer, acrylic polymer or copolymer, vinyl halide polymer, polyvinyl ether, polyvinylidene halide, polyacrylonitrile, polyvinyl ketone, polyvinyl aromatic, polyvinyl ester, copolymer of vinyl monomers, copolymer of vinyl monomers and olefins, polyamide, alkyd resin, polycarbonate, polyoxymethylene, polyimide, polyether, epoxy resin, polyurethane, rayon-triacetate, cellulose, cellulose acetate, cellulose butyrate, cellulose acetate butyrate, cellophane, cellulose nitrate, cellulose propionate, cellulose ethers, carboxymethyl cellulose, collagen, chitin, polylactic acid, polyglycolic acid, polylactic acid-polyethylene oxide copolymer, EPDM rubber, fluorosilicone, polyethylene glycol, polysaccharide, or phospholipid.

In particular embodiments, the therapeutic agent comprises an anti-thrombogenic agent, anti-angiogenesis agent, anti-proliferative agent, anti-restenosis agent, growth factor, radiochemical or antibiotic. In other embodiments, the therapeutic agent comprises an anti-restenosis agent. More specifically, in certain embodiments, the therapeutic agent comprises paclitaxel, sirolimus, everolimus, tacrolimus, or pimecrolimus. In some embodiments, the stent sidewall structure is balloon-expandable. In other embodiments, the stent comprises a metal stent.

In another embodiment, the present invention is directed to an intravascular stent designed for implantation into a blood vessel, such as a balloon expandable stent, comprising a stent sidewall structure with openings therein. The sidewall stent comprises a plurality of struts each having an outer surface (abluminal surface) and an inner surface (adluminal surface) opposite the outer surface. The stent includes a first coating composition, comprising a first polymer and a therapeutic agent, disposed on at least a portion of the outer surface of at least some of the struts. The first coating composition is not disposed on the inner surface of any of the struts. A second coating composition is disposed on the inner surface of at least some of the struts and on at least a portion of the first coating composition disposed on the outer surface of the struts. The second coating composition comprises a second polymer that is of different or less tackiness than the first polymer and the second coating composition is free of any therapeutic agent when applied to the inner surfaces and on the first coating composition disposed on the outer surface. In other embodiments, the first coating composition conforms to the outer surface of the struts to preserve the openings of the stent sidewall structure and the second coating composition conforms to the outer surface and inner surface of the struts to preserve the openings of the stent sidewall structure.

In certain embodiments, the struts comprise at least one side surface adjacent to the outer surface and the inner surface, which connects the inner surface and outer surface. The side surface is free of any coating composition. In other embodiments, the first coating composition is disposed on at least a portion of the side surface of at least some of the struts and the second coating composition is not disposed on the first coating composition that is disposed on the side surface of the struts. In other embodiments, the second coating composition is disposed on at least a portion of the side surface of at least some of the struts and the first coating composition is not disposed on the side surface of any of the struts. In alternative embodiments, the first coating composition is disposed on at least a portion of the side surface of at least some of the struts and the second coating composition is disposed on the first coating composition that is disposed on the side surface of the struts. In certain embodiments, the first coating composition conforms to the outer surface of the struts to preserve the openings of the stent sidewall structure and the second coating composition conforms to the outer surface and inner surface of the struts to preserve the openings of the stent sidewall structure.

In one embodiment, the second polymer is harder than the first polymer. In particular embodiments, the second polymer has a hardness of more than about or at least 40 A. In still other embodiments, the second polymer has a tackiness of less than about 50 g, such as about 3 g to about 30 g. In some embodiments, the first polymer has a tackiness of more than about 50 g. In certain embodiments, the first polymer and/or second polymer is biostable. In one embodiment, the polymer comprises a styrene-isobutylene copolymer, polyurethane, silicone, polyester, polyolefin, polyisobutylene, ethylene-alphaolefin copolymer, acrylic polymer or copolymer, vinyl halide polymer, polyvinyl ether, polyvinylidene halide, polyacrylonitrile, polyvinyl ketone, polyvinyl aromatic, polyvinyl ester, copolymer of vinyl monomers, copolymer of vinyl monomers and olefins, polyamide, alkyd resin, polycarbonate, polyoxymethylene, polyimide, polyether, epoxy resin, polyurethane, rayon-triacetate, cellulose, cellulose acetate, cellulose butyrate, cellulose acetate butyrate, cellophane, cellulose nitrate, cellulose propionate, cellulose ethers, carboxymethyl cellulose, collagen, chitin, polylactic acid, polyglycolic acid, polylactic acid-polyethylene oxide copolymer, EPDM rubber, fluorosilicone, polyethylene glycol, polysaccharide, or phospholipid.

In particular embodiments, the therapeutic agent comprises an anti-thrombogenic agent, anti-angiogenesis agent, anti-proliferative agent, anti-restenosis agent, growth factor, radiochemical or antibiotic. In certain embodiments, the therapeutic agent comprises an anti-restenosis agent. In certain embodiments, the therapeutic agent comprises paclitaxel, sirolimus, everolimus, tacrolimus, or pimecrolimus. In other embodiments, the stent sidewall structure is balloon-expandable. In still further embodiments, the stent comprises a metal stent.

In another embodiment, the invention is directed to an intravascular stent comprising a tubular stent sidewall structure with defined openings therein, wherein the stent sidewall structure comprises a plurality of struts each having an outer surface (abluminal surface) and an inner surface (adluminal surface) opposite the outer surface. There is a first coating composition disposed on at least a portion of the outer surface and inner surface of at least some of the struts. The first coating composition comprises a first polymer and is substantially free of any therapeutic agent. The stent comprises a second coating composition disposed on at least a portion of the first coating composition disposed on the outer surface of the struts. The second coating composition comprises a therapeutic agent and a second polymer that has more tackiness than the first polymer. The second coating composition is not disposed on the first coating composition disposed on the inner surface of the struts. In other embodiments, the first coating composition conforms to the outer surface and inner surface of the struts to preserve the openings of the stent sidewall structure and the second coating composition conforms to the outer surface of the struts to preserve the openings of the stent sidewall structure.

In certain embodiments, the struts comprise at least one side surface adjacent to the outer surface and the inner surface, which connects the inner surface and outer surface. In one embodiment, the side surface of the struts is free of any coating composition. In another embodiment, the first coating composition is disposed on at least a portion of the side surface of at least some of the struts and the second coating composition is not disposed on the first coating composition that is disposed on the side surface of the struts. In a particular embodiment, the second coating composition is disposed on at least a portion of the side surface of at least some of the struts and the first coating composition is not disposed on the side surface of any of the struts. In another embodiment, the first coating composition is disposed on at least a portion of the side surface of at least some of the struts and the second coating composition is disposed on the first coating composition that is disposed on the side surface of the struts. In certain embodiments, the first coating composition conforms to the outer surface and inner surface of the struts to preserve the openings of the stent sidewall structure and the second coating composition conforms to the outer surface of the struts to preserve the openings of the stent sidewall structure. In some embodiments, the first coating composition conforms to the outer surface, inner surface and side surface to preserve the openings of the stent sidewall structure and the second coating composition conforms to the inner surface to preserve the openings of the stent sidewall structure.

In particular embodiments, the second polymer is softer than the first polymer. In another embodiment, the second polymer has a hardness of less than about 40 A. In still other embodiments, the second polymer has a tackiness of greater than about 50 g, such as about 60 g to about 80 g or such as about 70 g. In some embodiments, the first polymer has a tackiness of less than about 50 g. In certain embodiments, the first polymer and/or the second polymer is biostable. In an embodiment, the first polymer comprises a styrene-isobutylene copolymer, polyurethane, silicone, polyester, polyolefin, polyisobutylene, ethylene-alphaolefin copolymer, acrylic polymer or copolymer, vinyl halide polymer, polyvinyl ether, polyvinylidene halide, polyacrylonitrile, polyvinyl ketone, polyvinyl aromatic, polyvinyl ester, copolymer of vinyl monomers, copolymer of vinyl monomers and olefins, polyamide, alkyd resin, polycarbonate, polyoxymethylene, polyimide, polyether, epoxy resin, polyurethane, rayon-triacetate, cellulose, cellulose acetate, cellulose butyrate, cellulose acetate butyrate, cellophane, cellulose nitrate, cellulose propionate, cellulose ethers, carboxymethyl cellulose, collagen, chitin, polylactic acid, polyglycolic acid, polylactic acid-polyethylene oxide copolymer, EPDM rubber, fluorosilicone, polyethylene glycol, polysaccharide, or phospholipid.

In certain embodiments, the first coating composition conforms to the outer surface and inner surface of the struts to preserve the openings of the stent sidewall structure and the second coating composition conforms to the inner surface of the struts to preserve the openings of the stent sidewall structure. In particular embodiments, the therapeutic agent comprises an anti-thrombogenic agent, anti-angiogenesis agent, anti-proliferative agent, anti-restensosis agent, growth factor, radiochemical or antibiotic. In alternative embodiments, the therapeutic agent comprises paclitaxel, sirolimus, everolimus, tacrolimus, or pimecrolimus.

In one embodiment, an intravascular balloon-expandable stent comprises a metal stent sidewall structure designed for implantation into a blood vessel of a patient, wherein the stent sidewall structure comprises a plurality of openings therein and struts each having an outer surface (abluminal surface) and an inner surface (adluminal surface) opposite the outer surface. There is a first coating composition disposed on at least a portion of the outer surface of at least some of the struts, wherein the first coating composition comprises a first biostable polymer and an anti-restenosis agent. The first coating composition is not disposed on the inner surface of any of the struts. The second coating composition is disposed on at least a portion of the inner surface of at least some of the struts and on at least a portion of the first coating composition disposed on the outer surface of the struts, wherein the second coating composition comprises a second biostable polymer that has less tackiness than the first polymer and is free of any therapeutic agent when applied to the outer and inner surfaces.

In some embodiments, the first coating composition conforms to the outer surface of the struts to preserve the openings of the stent sidewall structure and the second coating composition conforms to the outer surface and inner surface of the struts to preserve the openings of the stent sidewall structure. In certain embodiments, the second polymer is harder than the first polymer. In other embodiments, second polymer has a hardness of greater than about 40 A. In one embodiment, the second polymer has a tackiness of about 30 g. In a second embodiment, the therapeutic agent comprises an anti-thrombogenic agent, anti-angiogenesis agent, anti-proliferative agent, anti-restensosis agent, growth factor, radiochemical or antibiotic. In particular embodiments, the therapeutic agent comprises paclitaxel, sirolimus, everolimus, tacrolimus, or pimecrolimus.

In an alternative embodiment, an intravascular balloon-expandable stent comprises a metal stent sidewall structure designed for implantation into a blood vessel of a patient, wherein the stent sidewall structure comprises a plurality of openings therein and struts each having an outer surface (abluminal surface) and an inner surface (adluminal surface) opposite the outer surface. There is a first coating composition disposed on at least a portion of the outer surface and inner surface of at least some of the struts, wherein the first coating composition comprises a first biostable polymer and is free of any therapeutic agent when applied to the outer and inner surfaces. There is a second coating composition disposed on at least a portion of the first coating composition disposed on the outer surface of the struts, wherein the second coating composition comprises a therapeutic agent and a second biostable polymer that has more tackiness than the first polymer. The second coating composition is not disposed on the first coating composition disposed on the inner surface of the struts.

In particular embodiments, the first coating composition conforms to the outer surface and inner surface of the struts to preserve the openings of the stent sidewall structure and the second coating composition conforms to the outer surface of the struts to preserve the openings of the stent sidewall structure. In certain embodiments, the second polymer is softer than the first polymer. In other embodiments, the second polymer has a hardness of less than about 40 A. In some embodiments, the second polymer has a tackiness of about 50 g or more. In some embodiments, the second polymer has a tackiness of about 60 g to about 80 g. In an embodiment, the second polymer is a polyamide. In one embodiment, the therapeutic agent comprises an anti-thrombogenic, anti-angiogenic agent, anti-proliferative agent, anti-restenosis agent, growth factor, radiochemical or antibiotic. In other embodiments, the therapeutic agent comprises paclitaxel, sirolimus, everolimus, tacrolimus, or pimecrolimus.

### BRIEF DESCRIPTION OF THE DRAWINGS

FIG. 1 depicts a perspective view of an implantable intravascular stent, having a sidewall comprising a plurality of struts with an outer surface, an inner surface, and side surfaces.

FIG. 1A shows the outer surface, inner surface, and side surface of a strut of the implantable stent of FIG. 1.

FIG. 2 shows a cross-sectional view of an individual strut of a stent that has a first coating composition disposed on its outer surface.

FIG. 2A shows a cross-sectional view of an individual strut of a stent that has a first coating composition disposed on its outer surface and side surfaces.

FIG. 3 is a cross-sectional view of a strut of a stent with a first coating composition disposed on the outer surface and inner surface and a second coating composition disposed on the first coating composition that is disposed on the inner surface.

FIG. 3A is a cross-sectional view of a strut of a stent with a first coating composition disposed on the outer surface and inner surface and a second coating composition disposed on the side surfaces and the first coating composition that is disposed on the inner surface.

FIG. 3B is a cross-sectional view of a strut of a stent with a first coating composition disposed on the outer surface, inner surface and side surfaces, and a second coating composition disposed on the first coating composition that is disposed on the inner surface.

FIG. 3C is a cross-sectional view of a strut of a stent with a first coating composition disposed on the outer surface, inner surface and side surfaces, and a second coating composition disposed on the first coating composition that is disposed on the inner surface and side surfaces.

FIG. 4 is a cross-sectional view of a strut of a stent with a first coating composition disposed on the outer surface, and a second coating composition disposed on the inner surface and on the first coating composition that is disposed on the outer surface.

FIG. 4A is a cross-sectional view of a strut of a stent with a first coating composition disposed on the outer surface and side surfaces, and a second coating composition disposed on the inner surface and on the first coating composition that is disposed on the outer surface.

FIG. 4B is a cross-sectional view of a strut of a stent with a first coating composition disposed on the outer surface, and a second coating composition disposed on the inner surface and side surfaces, and on the first coating composition disposed on the outer surface.

FIG. 4C is a cross-sectional view of a strut of a stent with a first coating composition disposed on the outer surface and side surfaces, and a second coating composition disposed on the inner surface, and on the first coating composition disposed on the outer surface and side surfaces.

FIG. 5 is a cross-sectional view of a strut of a stent with a first coating composition disposed on the outer and inner surfaces, and a second coating composition disposed on the first coating composition that is disposed on the outer surface.

FIG. 5A is a cross-sectional view of a strut of a stent with a first coating composition disposed on the outer, inner and side surfaces, and a second coating composition disposed on the first coating composition that is disposed on the outer surface.

FIG. 5B is a cross-sectional view of a strut of a stent with a first coating composition disposed on the outer and inner surfaces, and a second coating composition disposed on the side surfaces and first coating composition that is disposed on the outer surface.

FIG. 5C is a cross-sectional view of a strut of a stent with a first coating composition disposed on the outer, inner and side surfaces, and a second coating composition disposed on the first coating composition that is disposed on the outer surface and side surfaces.

FIG. 6 shows an unexpanded stent disposed about a support.

### DESCRIPTION OF THE PREFERRED EMBODIMENTS

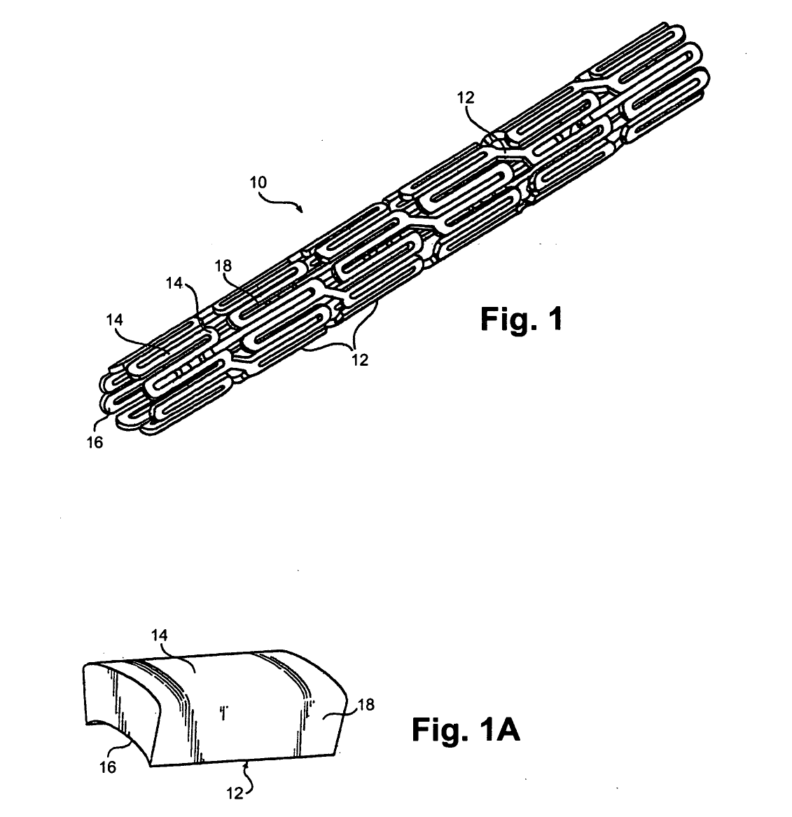

As shown in FIG. 1, the stents suitable for the present invention comprise a sidewall structure 10, such as a tubular sidewall. Such a sidewall 10 is preferably comprised of a plurality of struts 12. The struts 12 may be arranged in any suitable configuration. The struts 12 do not all have to have the same shape or geometric configuration. FIG. 1A is a cross-sectional view of a stent strut 12 depicted in FIG. 1. Generally, each individual strut 12 has an outer surface or abluminal surface 14, an inner surface or adluminal surface 16 opposite the outer surface 14, and at least one side surface 18. The outer surface 14 of the strut 12 is the surface that comes in direct contact with the body lumen wall when the stent is implanted. The outer surface 14 need not include only one flat surface or facet. Instead, it can be rounded, such as in the case of a wire strut 12, or have a number of facets. The inner surface 16 of the strut 12 is the surface that is opposite the outer surface 14 and generally faces the interior of the lumen. The two side surfaces 18 are the surfaces of the strut 12 that are adjacent to the inner surface 16 or outer surface 14. The side surface 18 connects the inner surface 16 and the outer surface 14. Like the outer surface 14, the inner surface 16 and side surface 18 can be rounded or have a number of facets.

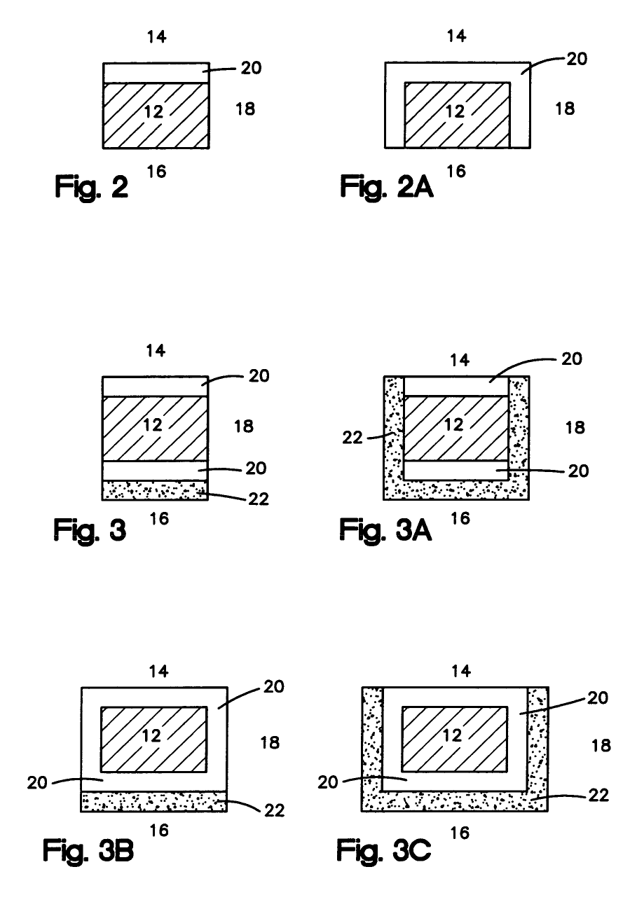

FIG. 2 shows a cross sectional view of a strut in one embodiment of the invention. As shown in FIG. 2, a first coating composition 20 is disposed on at least a portion of the outer surface 14 of a strut 12 of a stent. The first coating composition 20 comprises a therapeutic agent and a first polymer. The inner surface 16 and side surfaces 18 of the strut 12 are free of the first coating composition 20 or any coating composition.

In an alternative embodiment shown in FIG. 2A, the first coating composition 20 is disposed on the outer surface 14 and side surfaces 18 and the inner surface 16 is free of any coating composition. In other embodiments, the first coating composition 20 may be disposed on only one side surface 18.

FIG. 3 is a cross-sectional view of another embodiment of a stent having a coating composition disposed thereon. As shown in FIG. 3, a first coating composition 20 is disposed on the outer surface 14 and inner surface 16 of the strut. The first coating composition 20 comprises a therapeutic agent and a first polymer. A second coating composition 22 is disposed on at least a portion of the first coating composition 20 that is disposed on the inner surface 16. The second coating composition 22 is not disposed on the portion of the first coating composition 20 that is disposed on the outer surface 14. The second coating composition 22 comprises a second polymer that has a less or different tackiness than the first polymer and is substantially free of the therapeutic agent, i.e. contains less than 1% by weight of the second coating composition. In some embodiments, the second coating composition is free of any therapeutic agent.

The tackiness of a material can be measured by a texture analyzer and is generally considered to be the force required to separate the probe of the texture analyzer from the test surface as it is lifted from the surface. The tackiness of a polymer can be measured by a texture analyzer when the compressive force is about 50 grams, when the compressive force time is about 5 seconds, when the upward test speed is about 0.25 mm/s and/or the downward test speed is 0.020 mm/s. In certain embodiments, the more tacky polymers should have a tackiness of 50 g or more, such as about 60 g to about 80 g, e.g. about 70 g. The less tacky polymers should have a tackiness of 50 g or less such as about 3 g to about 30 g, e.g. about 16 g.

The hardness of a polymer can be measured by a 3 spring-loaded indenter which assesses hardness by computing the resistance of a material to indentation. The higher the number reported, the greater the resistance. The ASTM test method for hardness is ASTM D2240. The ISO test method for hardness is ISO 868. In some embodiments, it may be preferable that the less tacky polymer have a hardness of greater than about 40 A.

Another embodiment is shown in FIG. 3A which is similar to the embodiment in FIG. 3. In this embodiment, the second coating composition 22 is also disposed on the side surfaces 18 of the strut.

As shown in FIG. 3B, in another embodiment that is similar to the embodiment in FIG. 3, the first coating composition 20 is also disposed on the side surfaces 18.

The embodiment shown in FIG. 3C is similar to the one shown in FIG. 3. However, in this embodiment, the first coating composition 20 is also disposed on the side surfaces 18. The second coating composition 22 is disposed on the portion of the first coating composition 20 that is disposed on the inner surface 16 and the side surfaces 18.

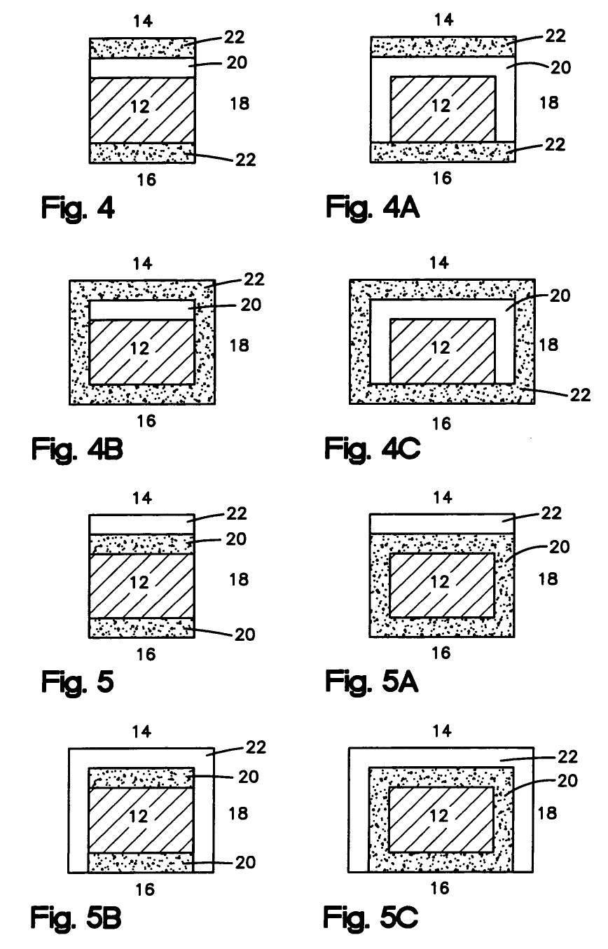

FIG. 4 shows another embodiment of the present invention. In this embodiment a first coating composition 20 is disposed on at least a portion of the outer surface 14 of the strut. The first coating composition 22 is not disposed on the inner surface 16. The first coating composition 20 comprises a therapeutic agent and a first polymer. A second coating composition 22 is disposed on the inner surface 16 and on the portion of the first coating composition 20 disposed on the outer surface 14. The second coating composition 22 comprises a second polymer that has a less or different tackiness than the first polymer and is substantially free of the therapeutic agent, i.e. contains less than about 1% by weight of the second coating composition. In some embodiments, the second coating composition is free of any therapeutic agent.

FIG. 4A shows an embodiment that is similar to the one shown in FIG. 4. In this embodiment, the first coating composition 20 is also disposed on the side surfaces 18. The embodiment in FIG. 4B is similar to that of FIG. 4 except that the second coating composition 22 is also disposed on the side surfaces of the strut. The embodiment in FIG. 4C is similar to that of FIG. 4B except that the first coating composition 20 is also disposed on the side surfaces 18.

In FIG. 5, the embodiment comprises a strut in which a first coating composition 20 is disposed on the outer surface 14 and on the inner surface 12 of the strut. The first coating composition 20 comprises a first polymer and is substantially free of a therapeutic agent, i.e. contains less than about 1% by weight of the second coating composition. A second coating composition 22 is disposed on at least a portion of the first coating composition 20 that is disposed on the outer surface 14. The second coating 22 comprises a therapeutic agent and a second polymer that is of different or greater tackiness than the first polymer of the first coating composition 20.

The embodiment in FIG. 5A is also similar to that of FIG. 5 except that the first coating composition 20 is disposed on the side surfaces 18 of the strut. FIG. 5B shows an embodiment that is similar to that of FIG. 5 except that the second coating composition 22 is also disposed on the side surfaces 18 of the strut. FIG. 5C shows an embodiment that is similar to that of FIG. 5B except that the first coating composition 20 is disposed on the side surfaces 18.

### A. Suitable Stents

The stents that are particularly suitable for the present invention include any kind of stent for medical purposes which is known to the skilled artisan. Suitable stents include, for example, vascular stents such as self expanding stents and balloon expandable stents. Examples of self expanding stents useful in the present invention are illustrated in U.S. Pat. Nos. 4,655,771 and 4,954,126 issued to Wallsten and 5,061,275 issued to Wallsten et al. Examples of appropriate balloon expandable stents are shown in U.S. Pat. No. 5,449,373 issued to Pinchasik et al. In certain embodiments, the stent comprises a stent sidewall structure with openings therein. When such stents are used, it is in some instances preferable to have the coating disposed on the stent to conform to the stent to preserve the openings of the sidewall structure. In preferred embodiments, the stent suitable for the present invention is an Express stent. More preferably, the Express stent is an Express™ stent or an Express2™ stent (Boston Scientific, Inc. Natick, Ma.).

Stents that are suitable for the present invention may be fabricated from metallic, ceramic, or polymers, or a combination thereof. Preferably, the materials are biocompatible. Metallic material is more preferable. Suitable metallic materials include metals and alloys based on titanium (such as nitinol, nickel titanium alloys, thermo memory alloy materials), stainless steel, tantalum, nickel chrome, or certain cobalt alloys including cobalt chromium nickel alloys such as Elgiloy® and Phynox®. Metallic materials also include clad composite filaments, such as those disclosed in WO 94/16646.

Suitable ceramic materials include, but are not limited to, oxides, carbides, or nitrides of the transition elements such as titanium oxides, hafnium oxides, iridiumoxides, chromium oxides, aluminum oxides, and zirconiumoxides. Silicon based materials, such as silica, may also be used. The polymer may be biostable. Also, the polymer may be biodegradable. Suitable polymers include, but are not limited to, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, and Teflon.

Polymers may be used for forming the stent in the present invention include without limitation isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for stents include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

Stents may also be made with non-polymers chemicals. Examples of useful non-polymers include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymers include cholesterol, glyceryl monostearate, glycerol tristearate, stearic acid, stearic anhydride, glyceryl monooleate, glyceryl monolinoleate, and acetylated monoglycerides.

### B. Suitable Therapeutic Agents

The term “therapeutic agent” encompasses biologically active material, and also genetic materials and biological materials. The therapeutic agents named herein include their analogs and derivatives. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, paclitaxel (as well as its derivatives, analogs or paclitaxel bound to proteins, e.g. Abraxane™) 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In another preferred embodiment, the therapeutic agent is paclitaxel or its analogs or derivatives (i.e. “paclitaxel”). In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolide agents such as sirolimus, pimerolimus, or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

Methods suitable for applying therapeutic agents to the devices of the present invention preferably do not alter or adversely impact the therapeutic properties of the therapeutic agent.

### C. Suitable Polymers

Polymers useful for forming the coatings should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, of styrene and isobutylene copolymers.

When the polymer is being applied to a part of the medical device, such as a stent, which undergoes mechanical challenges, e.g. expansion and contraction, the polymers are preferably selected from elastomeric polymers such as silicones (e.g. polysiloxanes and substituted polysiloxanes), polyurethanes, thermoplastic elastomers, ethylene vinyl acetate copolymers, polyolefin elastomers, and EPD rubbers. The polymer is selected to allow the coating to better adhere to the surface of the strut when the stent is subjected to forces or stress. Furthermore, although the coating can be formed by using a single type of polymer, various combinations of polymers can be employed.

Generally, when a hydrophilic therapeutic agent is used then a hydrophilic polymer having a greater affinity for the therapeutic agent than another material that is less hydrophilic is preferred. When a hydrophobic therapeutic agent is used then a hydrophobic polymer having a greater affinity for the therapeutic agent is preferred.

Examples of suitable hydrophobic polymers or monomers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by Ra SiO 4-a/2, where R is a monovalent substituted or unsubstituted hydrocarbon radical and the value of a is 1 or 2; and naturally occurring hydrophobic polymers such as rubber.

Examples of suitable hydrophilic polymers or monomers include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl(meth)acrylate and 2-hydroxypropyl(meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

Other suitable polymers include without limitation: polyurethanes, silicones (e.g., polysiloxanes and substituted polysiloxanes), and polyesters, styrene-isobutylene-copolymers. Other polymers which can be used include ones that can be dissolved and cured or polymerized on the medical device or polymers having relatively low melting points that can be blended with therapeutic agents. Additional suitable polymers include, but are not limited to, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, polyether block amides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPD (ethylene-propylene-diene) rubbers, fluoropolymers, fluorosilicones, polyethylene glycol, polysaccharides, phospholipids, and combinations of the foregoing.

### D. Methods for Forming the Coatings

The coating compositions can be prepared by dissolving or suspending a polymer and/or therapeutic agent in a solvent. Solvents that may be used to prepare coating compositions include ones which can dissolve or suspend the polymer and/or therapeutic agent in solution. Examples of suitable solvents include, but are not limited to, tetrahydrofuran, methylethylketone, chloroform, toluene, acetone, isooctane, 1,1,1, trichloroethane, dichloromethane, isopropanol, IPA, and mixture thereof.

The aforementioned coated medical devices can be made by applying coating compositions onto the surface of the medical device. Coating compositions can be applied by any method to a surface of a medical device or to another coating composition known by one skilled in the art. The different surfaces may be coated by the same or different methods. Suitable methods for applying the coating compositions to the medical devices include, but are not limited to, spray-coating, painting, rolling, electrostatic deposition, ink jet coating, dip coating, spin coating and a batch process such as air suspension, pan-coating or ultrasonic mist spraying, or a combination thereof.

In embodiments where a coating composition is to be applied to fewer than all the surfaces of the struts of a stent, such as on the stents described above, it is preferable to employ coating methods that selectively apply the coating composition. For instance, a first coating composition can be deposited onto a substrate. The substrate is preferably made from materials that has minimal adhesion the coating composition so that the coating composition can be easily removed and transferred to the surface. Then, the outer surface of the struts may be rolled over the coated substrate to transfer the coating composition to the outer surfaces of the struts.

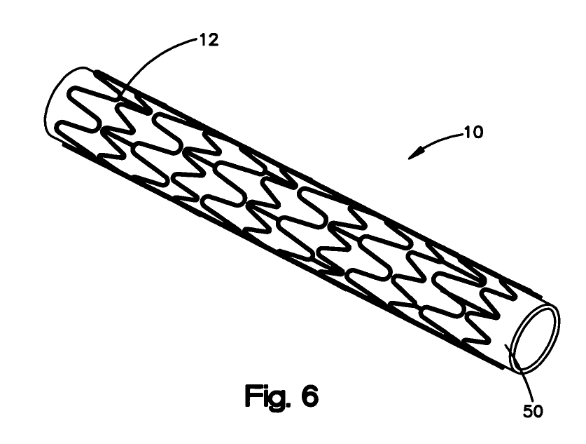

Also, it may be preferable to mask or cover the surface that is not to be coated with a particular coating composition. For instance in the embodiment in FIG. 2, to avoid having the first coating composition 20 disposed upon the inner surface 16 of the strut 12 and the side surfaces 18 of the strut 12, these surfaces can be masked. In one embodiment, the inner surface 16 can be masked by placing the stent 10 on a mandrel 50, such as that shown in FIG. 6. The inner surface 16 which is placed against the mandrel will not be exposed to a coating composition that is applied to the outer surface 14. For example, in one embodiment, the stent that is mounted on the mandrel may then be rolled over a substrate containing a coating composition to transfer the coating composition to the outer surface of the struts to form the embodiment in FIG. 2. Alternatively, the stent 10 can be placed on the mandrel 50 and the outer and side surfaces of the strut are spray-coating with the first composition 20 so that the embodiment of FIG. 2A is formed.

In an alternative embodiment, a bare stent can be dip coated with a material such as wax. In order to selectively coat particular portions of the stent, the wax coating can be ground off in selected locations, exposing the chosen locations of the stent struts. Subsequently, the stent can be spray coated, dipped, painted, rolled or by other means coated on the exposed locations. After the coating is complete, the wax on the remaining portions of the stent can be removed.

In embodiments where the coating composition is to be applied to the inner or side surfaces, the outer surface can be masked. For example, in the embodiments shown in FIGS. 3-3C, the outer surface is masked when the second coating composition is applied to the inner surface 16 and/or side surfaces 18. The outer surface 14 can be masked, for instance, by application of a protective wrap to that surface. The protective wrap is a material that would protect the coated surface from exposure to the coating applied to the opposing surface. Suitable material for this protective wrap include, for example, PTFE film, dyna-leap, Kapton®, or any other appropriate type of covering or wrapping material. The protective wrap preferably extends for the length of the stent, and is secured so that it does not unwrap. The protective wrap serves to protect the outer surface 14 from exposure to the second coating 22 composition as it is being applied to the inner surface 16. Thus, the protective wrap will protect an outer surface 14 that has been already coated from additional deposition of the coating to be applied to the inner surfaces 16 and side surfaces 18. After the inner surfaces 16 and side surfaces 18 of the struts 12 of the medical device have been coated, the wrap covering the outer surface 14 may be removed. A wrap can also be used to cover other surfaces, such as the inner and side surface, to prevent a coating composition from being disposed on such surfaces.

In embodiments, where the inner surface 16 and side surfaces 18 of stent 10 are to be coated, it may be preferable to use a spraying process. For example, a nozzle assembly may be used to spray a coating composition onto the inner surface. The nozzle assembly may be in the form of a cone that sprays the coating composition at an angle. The angle of the spray from the nozzles may need to be adjusted to ensure uniform thickness of the coating on the inner surface. Also, a nozzle assembly with small spray nozzles can be inserted into one end of the stent and moved through the stent until it extends past the opposite end of the stent. Preferably, the spray mist flow is started while the nozzle is still outside of the stent. This step places a coating composition on the inside surface and one side surface of the struts of the stent. The coating process may be repeated again. Preferably, the spray nozzle is inserted into the other end of the stent to coat the other side surface of the struts. By repeating the spraying from two directions, both side surfaces are coated with a coating composition.

Masking and selective coating techniques can be used to form the embodiments shown in the figures. For example, in the embodiments shown in FIGS. 3 and 3A, the side surfaces 18 of the strut 12 are masked and the first coating composition 20 is applied to the inner 16 and outer 14 surfaces. Thereafter, masking may be used to selectively dispose the second coating composition 22 on just the first coating composition 20 disposed on the inner surface 16 (as in FIG. 3) or on the side surfaces 18 and the first coating composition 20 disposed on the inner surface 16 (as in FIG. 3A). In the embodiments in FIGS. 3B-3C, the first coating composition 20 is applied to the outer 14, inner 16 and side 18 surfaces. Thereafter, masking may be used to selectively dispose the second coating composition 22 on just the first coating composition 20 disposed on the inner surface 16 (as in FIG. 3B) or on the first coating composition 20 disposed on the inner surface 16 and side surfaces 18 (as in FIG. 3C).

In the embodiments shown in FIGS. 4 and 4B, the inner surface 16 and the side surfaces 18 are masked and the first coating composition 20 is applied to the outer surface 14. Thereafter, masking may be used to selectively dispose the second coating composition 22 on the inner surface 16 and on the first coating composition 20 disposed on the outer surface 14 (as in FIG. 4). In the embodiments shown in FIGS. 4A and 4C, the inner surface 16 is masked and the first coating composition 20 is applied to the outer surface 14 and the side surfaces 18. Thereafter, masking may be used to selectively dispose the second coating composition 22 on the inner surface 16 and on the first coating composition 20 disposed on the outer surface 14 and (as in FIG. 4A).

In the embodiments shown in FIGS. 5 and 5B, the side surfaces 18 are masked and the first coating composition 20 is applied to the outer surface 14 and inner surface 16. Thereafter, masking may be used to selectively dispose the second coating composition 22 on the first coating composition 20 disposed on the outer surface 14 (as in FIG. 5) or on the side surfaces 18 and the first coating composition 20 disposed on the outer surface 14 (as in FIG. 5A). In the embodiments shown in FIGS. 5A and 5C, no masking is required for disposing the first coating composition 20, as the first coating composition 20 is disposed on the outer surface 14, inner surface 16, and the side surfaces 18. Thereafter, masking can be used to selectively dispose the second coating composition 22 on the first coating composition 20 disposed on the outer surface 14 (as in FIG. 5A) or on the first coating composition 20 disposed on the outer surface 14 and the side surfaces 18 (as in FIG. 5C).

After a coating composition has been applied, it can be cured. Curing is defined as the process of converting the polymeric material into the finished or useful state by the application of heat, vacuum, and/or chemical agents which induce physico-chemical changes. The applicable time and temperature for curing are determined by the particular polymer involved and particular therapeutic agent used, if any, as known by one skilled in the art. The coated medical devices may thereafter be subjected to a post-cure process wherein the medical devices are exposed to a low energy for stabilization of the coating. Also, after the medical device is coated, it preferably should be sterilized by methods of sterilization as known in the art.

In use, a coated medical device, such as an expandable stent, according to the present invention can be made to provide desired release profile of the therapeutic agent. The medical devices and stents of the present invention may be used for any appropriate medical procedure. Delivery of the medical device can be accomplished using methods well known to those skilled in the art, such as mounting the stent on an inflatable balloon disposed at the distal end of a delivery catheter.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed is:

1. An intravascular balloon-expandable stent comprising:
a metal stent sidewall structure designed for implantation into a blood vessel of a patient, wherein the sidewall structure comprises a plurality of struts and openings in the sidewall structure, and wherein the struts each have an outer surface and an inner surface opposite the outer surface;
a first coating composition disposed on at least a portion of the outer surface of at least one of the struts, wherein the first coating composition comprises a therapeutic agent and a first biostable polymer; and
wherein the inner surface of each of the struts is free of any coating composition; and wherein the first coating composition conforms to the outer surface of the at least one strut to preserve the openings of the stent sidewall structure.

2. The stent of claim 1, wherein the therapeutic agent comprises an anti-thrombogenic agent, anti-angiogenesis agent, anti-proliferative agent, anti-restensosis agent, growth factor, radiochemical or antibiotic.

3. The stent of claim 1, wherein the therapeutic agent comprises paclitaxel, sirolimus, everolimus, tacrolimus, or pimecrolimus.

4. An intravascular balloon-expandable stent comprising:
a metal stent sidewall structure designed for implantation into a blood vessel of a patient, wherein the sidewall structure comprises a plurality of struts and openings in the sidewall structure, and wherein the struts each have an outer surface and an inner surface opposite the outer surface;
a first coating composition disposed on at least a portion of the outer surface and the inner surface of at least one of the struts, wherein the first coating composition comprises a therapeutic agent and a first biostable polymer;
a second coating composition disposed on the first coating composition that is disposed on the inner surface of the at least one strut, wherein the second coating composition comprises a second biostable polymer that has less tackiness than the first polymer and is free of any therapeutic agent when applied to the first coating composition that is disposed on the inner surface; and
wherein the second coating composition is not disposed on the first coating composition that is disposed on the outer surface of the at least one strut.

5. The stent of claim 4, wherein the first coating composition conforms to the outer surface and inner surface of the at least one strut to preserve the openings of the sidewall structure and the second coating composition conforms to the inner surface of the at least one strut to preserve the openings of the sidewall structure.

6. The stent of claim 4, wherein the second polymer is harder than the first polymer.

7. The stent of claim 6, wherein second polymer has a hardness of more than about 40 A.

8. The stent of claim 4, wherein the second polymer has a tackiness of less than about 50 g.

9. The stent of claim 8, wherein the second polymer has a tackiness of about 3 g to about 30 g.

10. The stent of claim 4, wherein the second polymer comprises polyamide.

11. The stent of claim 4, wherein the therapeutic agent comprises an anti-thrombogenic agent, anti-angiogenesis agent, anti-proliferative agent, anti-restensosis agent, growth factor, radiochemical or antibiotic.

12. The stent of claim 4, wherein the therapeutic agent comprises paclitaxel, sirolimus, everolimus, tacrolimus, or pimecrolimus.

13. An intravascular balloon-expandable stent comprising:
a metal stent sidewall structure designed for implantation into a blood vessel of a patient, wherein the sidewall structure comprises a plurality of struts and openings in the sidewall structure, wherein the struts each having an outer surface and an inner surface opposite the outer surface;
a first coating composition disposed on at least a portion of the outer surface of at least one of the struts, wherein the first coating composition comprises a first biostable polymer and a therapeutic agent; and wherein the first coating composition is not disposed on the inner surface of any of the struts; and
a second coating composition disposed on at least a portion of the inner surface of the at least one strut and on at least a portion of the first coating composition disposed on the outer surface of the at least one strut, wherein the second coating composition comprises a second biostable polymer that has less tackiness than the first polymer and is free of any therapeutic agent when applied to the inner surfaces and on the first coating composition disposed on the outer surface.

14. The stent of claim 13, wherein the first coating composition conforms to the outer surface of the at least one strut to preserve the openings of the sidewall structure and the second coating composition conforms to the outer surface and inner surface of the at least one strut to preserve the openings of the sidewall structure.

15. The stent of claim 13, wherein the second polymer is harder than the first polymer.

16. The stent of claim 15, wherein second polymer has a hardness of at least about 40 A.

17. The stent of claim 13, wherein the second polymer has a tackiness of about less than 50 g.

18. The stent of claim 17, wherein the second polymer has a tackiness of about 3 g to about 30 g.

19. The stent of claim 13, wherein the second polymer comprises polyamide.

20. The stent of claim 13, wherein the therapeutic agent comprises an anti-thrombogenic agent, anti-angiogenesis agent, anti-proliferative agent, anti-restensosis agent, growth factor, radiochemical or antibiotic.

21. The stent of claim 13, wherein the therapeutic agent comprises paclitaxel, sirolimus, everolimus, tacrolimus, or pimecrolimus.

22. An intravascular balloon-expandable stent comprising:
a metal stent sidewall structure designed for implantation into a blood vessel of a patient, wherein the sidewall structure comprises a plurality of struts and openings in the sidewall structure, wherein the struts each have an outer surface and an inner surface opposite the outer surface;
a first coating composition disposed on at least a portion of the outer surface and inner surface of at least one of the struts, wherein the first coating composition comprises a first biostable polymer and is free of any therapeutic agent when applied to the outer and inner surfaces; and
a second coating composition disposed on at least a portion of the first coating composition disposed on the outer surface of the at least one strut, wherein the second coating composition comprises a therapeutic agent and a second biostable polymer that has more tackiness than the first polymer; and wherein the second coating composition is not disposed on the first coating composition disposed on the inner surface of the at least one strut.

23. The stent of claim 22, wherein the first coating composition conforms to the outer surface and inner surface of the at least one strut to preserve the openings of the stent sidewall structure and the second coating composition conforms to the outer surface of the at least one strut to preserve the openings of the stent sidewall structure.

24. The stent of claim 22, wherein the second polymer has a hardness that is less than that of the first polymer.

25. The stent of claim 24, wherein second polymer has a hardness of less than about 40 A.

26. The stent of claim 22, wherein the second polymer has a tackiness of about 50 g or more.

27. The stent of claim 26, wherein the second polymer has a tackiness of about 60 g to about 80 g.

28. The stent of claim 22, wherein the second polymer comprises polyamide.

29. The stent of claim 22, wherein the therapeutic agent comprises an anti-thrombogenic agent, anti-angiogenesis agent, anti-proliferative agent, anti-restensosis agent, growth factor, radiochemical or antibiotic.

30. The stent of claim 22, wherein the therapeutic agent comprises paclitaxel, sirolimus, everolimus, tacrolimus, or pimecrolimus.
